SEQUENCE LISTING (16 sequences)

SEQ ID NO: 1 - DNA, 15 bp, Artificial sequence
  Synthetic oligonucleotide
gcgcaacgggacgga

SEQ ID NO: 2 - DNA, 20 bp, Artificial sequence
  Synthetic oligonucleotide
gtaaagcttcacggggtctt

SEQ ID NO: 3 - DNA, 20 bp, Artificial sequence
  Synthetic oligonucleotide
gactcggtgaaatccaggta

SEQ ID NO: 4 - DNA, 24 bp, Artificial sequence
  Synthetic oligonucleotide
atggtggtgttttgatcaatatta

SEQ ID NO: 5 - DNA, 31 bp, Artificial sequence
  Synthetic oligonucleotide
ggctgactgcggactcggtgaaatccaggta

SEQ ID NO: 6 - DNA, 31 bp, Artificial sequence
  Synthetic oligonucleotide
cttcagcaatcctctacatggtggtgttttg

SEQ ID NO: 7 - DNA, 18 bp, Artificial sequence
  Synthetic oligonucleotide
cttcagcaatcctctaca

SEQ ID NO: 8 - DNA, 18 bp, Artificial sequence
  Synthetic oligonucleotide
ggctgactgcggactcgg

SEQ ID NO: 9 - DNA, 35 bp, Artificial sequence
  Synthetic oligonucleotide
gcgcaacgggacggaaagaccccgtgaagctttac

SEQ ID NO: 10 - DNA, 20 bp, Artificial sequence
  Synthetic oligonucleotide
ttctggggcacttcgaaatg

SEQ ID NO: 11 - DNA, 35 bp, Artificial sequence
  Synthetic oligonucleotide
cgcgttgccctgcctttctggggcacttcgaaatg

SEQ ID NO: 12 - DNA, 35 bp, Artificial sequence
  Synthetic oligonucleotide
gcgcaacgggacggcaagaccccgtgaagctttac

SEQ ID NO: 13 - DNA, 24 bp, Artificial sequence
  Synthetic oligonucleotide
agggcagaagcgcaacgggacgga

SEQ ID NO: 14 - DNA, 30 bp, Artificial sequence
  Synthetic oligonucleotide
ttctggggcacttcgaaatggcgtgactgg

SEQ ID NO: 15 - DNA, 54 bp, Artificial sequence
  Synthetic oligonucleotide
agggcagaagcgcaacgggacggaaagaccccgtgaagctttaccgcactgacc

SEQ ID NO: 16 - DNA, 54 bp, Artificial sequence
  Synthetic oligonucleotide
tcccgtcttcgcgttgccctgcctttctggggcacttcgaaatggcgtgactgg

(19) United States
(12) Patent Application Publication
(10) Pub. No.: US 2020/0208207 A1
(43) Pub. Date: Jul. 2, 2020

(54) METHODS FOR DETECTING VARIANT NUCLEOTIDES

(71) Applicant: LUMINEX CORPORATION, Austin, TX (US)

(72) Inventors: T.S. RAMASUBRAMANIAN, Austin, TX (US); Lindsay HILL-BATORSKI, Austin, TX (US); M. Maggie O'MEARA, Austin, TX (US); Victoria BROWNING, Austin, TX (US)

(73) Assignee: LUMINEX CORPORATION, Austin, TX (US)

(21) Appl. No.: 16/720,065

(22) Filed: Dec. 19, 2019

Related U.S. Application Data
(60) Provisional application No. 62/786,137, filed on Dec. 28, 2018.

(51) Int. Cl. C12Q 1/6853

(52) U.S. Cl. CPC C12Q 1/6853

(57) ABSTRACT

Methods and compositions are provided for determining the presence of a wild type or variant nucleotide at a position in a nucleic acid molecule of interest.

This application claims the benefit of U.S. Provisional Patent Application No. 62/786,137, filed Dec. 28, 2018, the entirety of which is incorporated herein by reference.

### BACKGROUND OF THE INVENTION

### Field of the Invention

The present invention relates generally to the field of molecular biology. More particularly, it concerns the detection of nucleic acids.

### Description of Related Art

Known methods of detecting variant nucleotides or SNPS using real time amplification include the use of (i) labeled allele-specific primers which only efficiently amplify targets having perfectly complementary sequences at the 3′ end of the primers and (ii) common primers that amplify the regions of interest containing the potential variant nucleotide(s) and detection and identification of amplified targets using probe-based methods. These methods often require either multiple allele-specific primers to ensure all possible variant sequences are amplified or multiple allele-specific probes to detect all possible alleles. In a multiplex system where variants at multiple locations within a single gene are being investigated, the complexity of designing multiple primers or probes to ensure all variant nucleotides are amplified and detected in a single reaction chamber can be challenging. This is especially true if real-time detection is desired, since there is a limit to the number of distinguishable fluorophores that are available for use in most real-time detection systems. These methods are also not generally amenable to distinguishing closely located variant nucleotides, such as those that occur at adjacent positions in a target nucleic acid sequence or within 15-20 nucleotides of each other on a target nucleic acid sequence, because primers and probes compete for the same binding regions. There is a need for an assay method that can distinguish wild-type alleles from variant alleles in a real time reaction that does not require different reagents for detecting different alleles and that can be used to interrogate target nucleic acids that have adjacent or closely located (within 15-20 nucleotides) variant nucleotides. Furthermore, it would be advantageous if universal reagents could be used to detect all possible variations at a particular nucleotide and if melt analysis was not required to distinguish amplification products containing a wild-type nucleotide at a position of interest from a variant nucleotide.

### SUMMARY OF THE INVENTION

The present invention relates to methods for detecting variant nucleotides in a nucleic acid of interest. In particular, embodiments of the present invention provide methods suitable for detecting multiple different variant nucleotides at a position of interest using a limited number of primers and probes.

In a first embodiment, a method for determining the presence of a wild type or variant nucleotide at a position of interest in a target nucleic acid sequence is provided, the target nucleic acid sequence having first and second regions and the position of interest being located in the first region, the method comprising the steps of a) providing a first primer pair capable of specific amplification of the first region of the target nucleic acid sequence if present, to form a first amplicon, wherein one primer of the pair has a 3′ terminal nucleotide that is complementary to the wild type nucleotide at the position of interest and wherein the first amplicon is labeled with a first signal generating label coupled to one of the primers of the first pair of primers; b) providing a second primer pair capable of specific amplification of the second region of the target nucleic acid sequence, if present, to form a second amplicon, wherein the second amplicon is labeled with a second signal-generating label coupled to one of the primers of the second pair of primers; c) forming a reaction mixture comprising the first and second pairs of primers, and the target nucleic acid under conditions for nucleic acid amplification; d) measuring first and second signals from each of the first and second labels as amplification proceeds and calculating a cycle threshold (Ct) value associated with each of the first and second signals; e) comparing the Ct values associated with the first and second signals; and f) determining the presence of a wild-type nucleotide at the position of interest if the difference between the Ct values associated with the first and second signals is less than or equal to a predetermined threshold or determining the presence of a variant nucleotide at the position of interest if the difference between the Ct values associated with the first and second signals is greater than the predetermined threshold. In certain embodiments, the first and second regions of the target nucleic acid partially overlap and in other embodiments the first and second regions of the target nucleic acid do not overlap. In some embodiments, the first and second regions of the target nucleic acid are within 500 nucleotides of each other, or within 300 nucleotides of each other, or within 200 nucleotides of each other, or within 150 nucleotides of each other, or within 100 nucleotides of each other, or within 80 nucleotides of each other. In certain embodiments, the first and second signal-generating labels are distinguishable fluorophores. In certain embodiments the first and second signal generating labels are coupled to a non-standard base at a 5′ end of each primer. In certain such embodiments, the non-standard base is one of iso-C or iso-G. In certain embodiments, amplification results in the incorporation of a complementary non-standard base opposite the non-standard base of each primer. In certain such embodiments, the first and second signal-generating labels are distinguishable fluorophores and the complementary non-standard base is coupled to a quencher. In certain embodiments, the predetermined threshold is determined as the difference in Ct values associated with the first and second signals from a target nucleic acid having a wild-type nucleotide at the position of interest.

In another embodiment, a method of determining the presence of a wild-type or variant nucleotide at each of first and second positions of interest in a target nucleic acid, wherein the first and second positions of interest are within 15-20 nucleotides of each other is provided, the method comprising the steps of a) providing a first primer pair capable of specific amplification of a first portion of the target nucleic acid to form a first amplicon, wherein one primer of the pair has a 3′ terminal nucleotide that is complementary to the wild-type nucleotide at the first position of interest and wherein the first amplicon is labeled with a first signal-generating label coupled to one of the primers of the first pair of primers; b) providing a second primer pair capable of specific amplification of a second portion of the target nucleic acid to form a second amplicon, wherein one primer of the second primer pair has a 3′ terminal nucleotide that is complementary to the complement of the wild-type nucleotide at the second position of interest, and wherein the second amplicon is labeled with a second signal-generating label coupled to one of the primers of the second pair of primers; c) providing a third primer pair capable of specific amplification of a third portion of the target nucleic acid, if present, to form a third amplicon, wherein the third amplicon does not overlap with the first or second amplicon, and wherein the third amplicon is labeled with a third signal-generating label coupled to one of the primers of the third pair of primers; d) forming a reaction mixture comprising the first, second and third pairs of primers, and the target nucleic acid under conditions for nucleic acid amplification; e) measuring signal from each of the first, second and third labels as amplification proceeds and calculating a Ct value associated with each of the first, second and third signals; f) comparing the Ct values associated with the first and third signals and comparing the Ct values associated with the second and third signals; and g) determining the presence of the wild-type nucleotide at the first position of interest if the difference between the Ct values associated with the first and third signals is less than or equal to a first predetermined threshold, determining the presence a variant nucleotide at the first position of interest if the difference between the Ct values associated with the first and third signals is greater than the first predetermined threshold, determining the presence of the wild type nucleotide at the second position of interest if the difference between the Ct values associated with the second and third labels is less than or equal to a second predetermined threshold or determining the presence of a variant nucleotide at the second position of interest if the difference between the Ct values associated with the second and third labels is greater than the second predetermined threshold. In certain embodiments, the first and second portions of the target nucleic acid overlap. In certain embodiments, the first, second and third signal-generating labels are distinguishable fluorophores. In certain embodiments, the first, second and third signal-generating labels are coupled to a non-standard base at a 5′ end of each primer. In some embodiments, the non-standard base is one of isoC or isoG. In certain embodiments, amplification results in the incorporation of a complementary non-standard base opposite the non-standard base of each primer. In some such embodiments, the first, second and third signal-generating labels are distinguishable fluorophores, and the complementary non-standard base is coupled to a quencher. In certain embodiments, the first predetermined threshold is determined as the difference in Ct values associated with the first and third signals from a target nucleic acid having a wild type nucleotide at the first position of interest and the second predetermined threshold is determined as the difference in Ct values associated with the second and third signals from a target nucleic acid having a wild-type nucleotide at the second position of interest.

Another embodiment provides a method of determining the presence of a wild type or variant nucleotide at a position of interest in a target nucleic acid, the target nucleic acid having first and second regions and the position of interest being located in the first region, the method comprising the steps of a) providing a first primer pair capable of specific amplification of the first region of the target nucleic acid to form a first amplicon, wherein one primer of the pair has a 3′ terminal nucleotide that is complementary to the wild-type nucleotide at the position of interest, and one primer of the pair has a 5′portion and a 3′ portion, the 5′ portion comprising a first unique tag that is not complementary to the target nucleic acid and a 3′ portion that specifically hybridizes to the target nucleic acid sequence; b) providing a second primer pair capable of specific amplification of the second region of the target nucleic acid to form a second amplicon, wherein one primer of the pair has a 5′portion and a 3′ portion, the 5′ portion comprising a second unique tag that is not complementary to the target nucleic acid and the 3′ portion being complementary to the target nucleic acid; c) providing a first signal-generating probe sufficiently complementary to the complement of the first unique tag to specifically hybridize thereto; d) providing a second signal generating probe sufficiently complementary to the complement of the second unique tag to specifically hybridize thereto, wherein the signals from the first and second signal generating probes are distinguishable; e) forming a reaction mixture comprising the first and second primer pairs, the first and second signal-generating probes and the target nucleic acid under conditions for nucleic acid amplification; f) measuring first and second signals from each of the first and second signal-generating probes as amplification proceeds and calculating first and second Ct values associated with the first and second signals respectively; and g) comparing the first and second Ct values and determining the presence of a wild type nucleotide at the position of interest if the difference between the first and second Ct values is less than or equal to a predetermined threshold or determining the presence of a variant nucleotide at the position of interest if the difference between the first and second Ct values is greater than the predetermined threshold. In certain embodiments, the first and second probes have a sequence that is the same as the sequence of the first and second tagged primers respectively. In certain other embodiments, the first and second probes have a sequence that is only partially complementary to the complement of the first and second tagged primers respectively. In certain embodiments, the primer having the first unique 5′ tag has a 3′ terminal nucleotide complementary to the wild-type nucleotide at the position of interest. In other embodiments, the primer having the first unique 5′ tag is not the same primer as the primer having a 3′ terminal nucleotide complementary to the wild-type nucleotide at the position of interest. In certain embodiments, each of the first and second signal-generating probes are capable of generating a signal in the presence of target nucleic acid that is different from the signal generated in the absence of target. In certain embodiments, each of the signal generating probes is labeled with a fluorophore and a quencher, and the fluorophores of the first and second signal-generating probes are distinguishable. In certain embodiments, the first and second regions of the target nucleic acid partially overlap. In other embodiments, the first and second regions of the target nucleic acid do not overlap. In certain embodiments, the first and second regions of the target nucleic acid are within 500 nucleotides of each other, or within 300 nucleotides of each other, or within 200 nucleotides of each other, or within 150 nucleotides of each other, or within 100 nucleotides of each other, or within 80 nucleotides of each other. In certain embodiments, the predetermined threshold is determined as the difference in Ct values associated with the first and second signals from a target nucleic acid having a wild-type nucleotide at the position of interest.

In another embodiment, a method of determining the presence of a wild-type or variant nucleotide at first and second positions of interest in a target nucleic acid sequence, the first and second positions of interest being within 15-20 nucleotides of each other is provided, the method comprising the steps of a) providing a first primer pair capable of specific amplification of a first portion of the target nucleic acid to form a first amplicon, wherein one primer of the pair has a 3′ terminal nucleotide that is complementary to the wild-type nucleotide at the first position of interest, and one primer of the pair has a 5′portion and a 3′ portion, the 5 portion comprising a first unique tag that is not complementary to the target nucleic acid and a 3′ portion that specifically hybridizes to the first portion of the target nucleic acid sequence; b) providing a second primer pair capable of specific amplification of a second portion of the target nucleic acid to form a second amplicon, wherein one primer of the pair has a 3′ terminal nucleotide that is the same as the wild-type nucleotide at the second position of interest, and one primer of the pair has a 5′portion and a 3′ portion, the 5′ portion comprising a second unique tag that is not complementary to the target nucleic acid and the 3′ portion being complementary to the second portion of the target nucleic acid; c) providing a third primer pair capable of specific amplification of a third portion of the target nucleic acid sequence, one primer of the pair having a 5′ portion comprising a third unique tag that is not complementary to the target nucleic acid sequence and a 3′ portion being complementary to the third portion of target nucleic acid and the third portion of the target nucleic acid sequence being non-overlapping with the first and second portions; d) providing a first signal-generating probe, the first signal generating probe being sufficiently complementary to the first unique tag to specifically hybridize thereto; e) providing a second signal-generating probe, the second signal generating probe being sufficiently complementary to the second unique tag to specifically hybridize thereto; f) providing a third signal-generating probe, the third signal generating probe being sufficiently complementary to the third unique tag to specifically hybridize thereto, wherein the first, second and third signals of the first, second and third signal generating probes are distinguishable; g) forming a reaction mixture comprising the first, second and third primer pairs and the first, second and third signal-generating probes and the target nucleic acid under conditions for nucleic acid amplification; h) measuring first, second and third signals from each of the first, second and third signal-generating probes while amplification proceeds and calculating a Ct value associated with each of the first, second and third signals; i) comparing the first and third Ct values and determining the presence of a wild-type nucleotide at the first position of interest if the difference between Ct values from the first and third signal-generating probes is less than or equal to a first predetermined threshold, and determining the presence of a variant nucleotide at the first position of interest if the difference between Ct values from the first and third signal generating probes is greater than the first predetermined threshold; and j) comparing the second and third Ct values and determining the presence of a wild-type nucleotide at the position of interest if the difference between Ct values from the second and third signal generating probes is less than or equal to a second predetermined threshold and determining the presence of a variant nucleotide at the second position of interest if the difference between Ct values from the second and third signal-generating probes is greater than the second predetermined threshold. In certain embodiments, the first, second and third probes have a sequence that is the same as the sequence of the first, second and third tagged primers respectively. In certain other embodiments, the first, second and third probes have a sequence that is only partially complementary to the complement of the first, second and third tagged primers respectively. In certain embodiments, for the first primer set, the primer having the first unique 5′ tag has a 3′ terminal nucleotide complementary to the wild-type nucleotide at the first position of interest, and for the second primer set, the primer having the second unique 5′ tag has a 3′ terminal nucleotide complementary to the wild-type nucleotide at the second position of interest. In other embodiments, the primer having the first unique 5′ tag of the first primer set is not the same primer as the primer having a 3′ terminal nucleotide complementary to the wild type nucleotide at the first position of interest, and the primer having the second unique 5′ tag of the second primer set is not the same primer as the primer having a 3′ terminal nucleotide complementary to the wild-type nucleotide at the second position of interest. In certain embodiments, the first, second and third signal-generating probes are capable of generating a signal in the presence of target nucleic acid that is different than the signal generated in the absence of target. In certain embodiments, each of the signal generating probes is labeled with a fluorophore and a quencher, and the fluorophores of the first, second and third signal-generating probes are distinguishable. In certain embodiments, the first and second regions of the target nucleic acid are within 500 nucleotides of each other, or within 300 nucleotides of each other, or within 200 nucleotides of each other, or within 150 nucleotides of each other, or within 100 nucleotides of each other, or within 80 nucleotides of each other. In certain embodiments, the first predetermined threshold is determined as the difference in Ct values associated with the first and third signals from a target nucleic acid having a wild type nucleotide at the first position of interest and the second predetermined threshold is determined as the difference in Ct values associated with the second and third signals from a target nucleic acid having a wild-type nucleotide at the second position of interest.

Another embodiment provides a method of determining the presence of a wild-type or variant nucleotide at a position of interest in a target nucleic acid, the target nucleic acid having first and second regions and the nucleotide of interest being located in the first region, the method comprising the steps of a) providing a first primer pair capable of specific amplification of the first region of the target nucleic acid to form a first amplicon, wherein one primer of the pair is an allele-specific primer and has a Tm that is at least 3° C. degrees higher when hybridized to a target nucleic acid having a wild-type nucleotide at the position of interest than when hybridized to a target nucleic acid having a variant nucleotide at the position of interest, and wherein the first amplicon is labeled with a first signal-generating label coupled to one of the primers of the first pair of primers; b) providing a second primer pair capable of specific amplification of the second region of the target nucleic acid to form a second amplicon, wherein the second amplicon is labeled with a second signal-generating label coupled to one of the primers of the second pair of primers; c) forming a reaction mixture comprising the first and second pairs of primers, and the target nucleic acid under conditions for nucleic acid amplification; d) measuring first and second signals from each of the first and second signal-generating labels as amplification proceeds and calculating first and second Ct values associated with each of the first and second signals; and e) comparing the first and second Ct values and determining the presence of a wild-type nucleotide at the position of interest if the difference between the first and second Ct values is less than or equal to a predetermined threshold or determining the presence of a variant nucleotide at the position of interest if the difference between the first and second Ct values is greater than the predetermined threshold. In certain embodiments, the allele-specific primer hybridizes to the target nucleic acid such that the position of interest corresponds to a 3′ terminal nucleotide of the primer. In other embodiments, the allele-specific primer hybridizes to the target nucleic acid such that the position of interest corresponds to the nucleotide immediately upstream of a 3′ terminal nucleotide of the primer. In other embodiments the allele-specific primer hybridizes to the target nucleic acid such that the position of interest corresponds to the nucleotide two positions upstream of a 3′ terminal nucleotide of the primer. In some embodiments, the first and second regions of the target nucleic acid partially overlap. In other embodiments, the first and second regions of the target nucleic acid do not overlap. In certain embodiments, the first and second regions of the target nucleic acid are within 500 nucleotides of each other, or within 300 nucleotides of each other, or within 200 nucleotides of each other, or within 150 nucleotides of each other, or within 100 nucleotides of each other, or within 80 nucleotides of each other. In certain embodiments, the first and second signal-generating labels are distinguishable fluorophores. In certain embodiments, the first and second signal-generating labels are coupled to a non-standard base at a 5′ end of each primer. In certain aspects, the non-standard base is one of isoC or isoG. In certain embodiments, amplification results in the incorporation of a complementary non-standard base opposite the non-standard base of each primer. In certain embodiments, the first and second signal-generating labels are distinguishable fluorophores, and the complementary non-standard base is coupled to a quencher. In certain embodiments, the predetermined threshold is determined as the difference in Ct values associated with the first and second signals from a target nucleic acid having a wild-type nucleotide at the position of interest.

Certain aspects of the embodiments concern the use of at least one non-natural nucleotide. In some aspects, the non-natural nucleotide is an isobase, such as iso-guanine (isoG) or iso-cytosine (isoC). In certain aspects, the at least one non-natural nucleotide or the quencher-labeled non-natural nucleotide may be isoG and the other may be isoC.

Various probes, compositions, and methods disclosed herein comprise use of a reporter. A reporter or labeling agent is a molecule that facilitates the detection of a molecule (e.g., a nucleic acid sequence) to which it is attached. Numerous reporter molecules that may be used to label nucleic acids are known. Direct reporter molecules include fluorophores, chromophores, and radiophores. Non-limiting examples of fluorophores include, a red fluorescent squaraine dye such as 2,4-Bis[1,3,3-trimethyl-2-indolinylidenemethyl]cyclobutenediylium-1,3-dio-xolate, an infrared dye such as 2,4 Bis[3,3-dimethyl-2-(1H-benz[e]indolinylidenemethyl)]cyclobutenediylium-1,-3-dioxolate, or an orange fluorescent squaraine dye such as 2,4-Bis[3,5-dimethyl-2-pyrrolyl]cyclobutenediylium-1,3-diololate. Additional non-limiting examples of fluorophores include quantum dots, Alexa Fluor™ dyes, AMCA, BODIPY™ 630/650, BODIPY™ 650/665, BODIPY™-FL, BODIPY™-R6G, BODIPY™-TMR, BODIPY™-TRX, Cascade Blue™, CyDye™, including but not limited to Cy2™, Cy3™, and Cy5™, a DNA intercalating dye, 6-FAM™, Fluorescein, HEX™, 6-JOE, Oregon Green™ 488, Oregon Green™ 500, Oregon Green™ 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, or Texas Red™. A signal amplification reagent, such as tyramide (PerkinElmer), may be used to enhance the fluorescence signal. Indirect reporter molecules include biotin, which must be bound to another molecule such as streptavidin-phycoerythrin for detection. Pairs of labels, such as fluorescence resonance energy transfer pairs or dye-quencher pairs, may also be employed.

Labeled amplification products may be labeled directly or indirectly. Direct labeling may be achieved by, for example, using labeled primers, using labeled dNTPs, using labeled nucleic acid intercalating agents, or combinations of the above. Indirect labeling may be achieved by, for example, hybridizing a labeled probe to the amplification product.

The probes and methods disclosed herein may be employed in the detection of target nucleic acid sequences. The target nucleic acid sequence may be any sequence of interest. The sample containing the target nucleic acid sequence may be any sample that contains nucleic acids. In certain aspects of the invention the sample is, for example, a subject who is being screened for the presence or absence of one or more genetic mutations or polymorphisms. In another aspect of the invention the sample may be from a subject who is being tested for the presence or absence of a pathogen. Where the sample is obtained from a subject, it may be obtained by methods known to those in the art such as aspiration, biopsy, swabbing, venipuncture, spinal tap, fecal sample, or urine sample. In some aspects of the invention, the sample is an environmental sample such as a water, soil, or air sample. In other aspects of the invention, the sample is from a plant, bacteria, virus, fungi, protozoan, or metazoan.

Various methods disclosed herein use PCR amplification. Each amplification cycle has three phases: a denaturing phase, a primer annealing phase, and a primer extension phase. In practice however, thermal cyclers can be programmed to cycle between denaturation and primer annealing phases only. The amplification cycle can be repeated until the desired amount of amplification product is produced. Typically, the amplification cycle is repeated between about 10 to 40 times. For real-time PCR, detection of the amplification products will typically be done after each amplification cycle. Although in certain aspects of the invention, detection of the amplification products may be done after every second, third, fourth, or fifth amplification cycle. Detection may also be done such that as few as 2 or more amplification cycles are analyzed or detected. The amplification cycle may be performed in the same chamber in which the detection of the amplification occurs, in which case this chamber would need to comprise a heating element so the temperature in the chamber can be adjusted for the denaturing phase, primer annealing phase, and a primer extension phase of the amplification cycle. The heating element would typically be under the control of a processor. The amplification cycle may, however, be performed in a different chamber from the chamber in which detection of the amplification occurs, in which case the “amplification” chamber would need to comprise a heating element, but the “detection” or “imaging” chamber would not be required to have a heating element. Where amplification and detection occur in separate chambers, the fluid in which the amplification reaction occurs may be transferred between the chambers by, for example, a pump or piston. The pump or piston may be under the control of a processor. Alternatively, the fluid may be transferred between the chambers manually using, for example, a pipette.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B—A non-limiting exemplary schematic showing a method of the embodiments.

FIG. 1C-D—A non-limiting exemplary schematic showing a method of the embodiments.

FIG. 2A-B—A non-limiting exemplary schematic showing a method of the embodiments.

FIG. 2C-D—A non-limiting exemplary schematic showing a method of the embodiments.

FIG. 3A-B—A non-limiting exemplary schematic showing a method of the embodiments.

Sequences in the figures:

GCGCAACGGGACGGA = SEQ ID NO: 1;
GCGCAACGGGACGGAAAGACCCCGTGAAGCTTTAC = SEQ ID NO:
9;
TTCTGGGGCACTTCGAAATG = SEQ ID NO: 10;
CGCGTTGCCCTGCCTTTCTGGGGCACTTCGAAATG = SEQ ID NO:
11;
GCGCAACGGGACGGCAAGACCCCGTGAAGCTTTAC = SEQ ID NO:
12;
AGGGCAGAAGCGCAACGGGACGGA = SEQ ID NO: 13;
TTCTGGGGCACTTCGAAATGGCGTGACTGG = SEQ ID NO: 14;
AGGGCAGAAGCGCAACGGGACGGAAAGACCCCGTGAAGCTTTACCGCACT
GACC = SEQ ID NO: 15;
TCCCGTCTTCGCGTTGCCCTGCCTTTCTGGGGCACTTCGAAATGGCGTGA
CTGG = SEQ ID NO: 16.

### DESCRIPTION OF ILLUSTRATIVE EMBODIMENTS

The method presented herein for distinguishing wild-type nucleotides from variant nucleotides is independent of the specific variant nucleotide at a position of interest within the target nucleic acid sequence, since the method utilizes reagents that detect only wild-type sequences. Nevertheless, the assay design permits a user to distinguish a wild-type nucleotide from a variant nucleotide at a specific position or at 2 or more positions of interest within the target nucleic acid sequence. The method thus enables the detection of a variant nucleotide regardless of the actual nucleotide at that position and relies on determination and analysis of cycle threshold (Ct) values of target nucleic acid amplification.

The method utilizes methods of relative quantification of amplification products whereby the differences in Real-Time PCR efficiency can be measured. While the method refers to the determination of Ct values, a person skilled in the art would recognize that that any derivative method or baseline call reported as Ct (Cycle threshold), Cp (Crossing point), TOP (Take-off point) or Cq (Quantification cycle) values could be utilized in the method to determine the PCR cycle at which the amplification of the target becomes detectable. The Cq or Ct value represents the cycle number based on the point where the measured fluorescence rises above the background fluorescence to cross a predetermined fluorescence background threshold value. Multiple approaches may be used to determine this cycle number such as: 1) A second derivative method wherein a mathematical transformation of the amplification curve to a second derivative curve provides Cp value at the peak height; 2) 5 point rolling method where the standard deviation over a sliding window of length across neighboring elements provides a Cp peak; 3) 5 point Mean Standard Deviation method wherein a rolling 5 point fluorescence mean and standard deviation is used to calculate the number of SDs that the next cycle's fluorescence is away from the mean of the previous 5 cycles that identifies the Cp value as the one where the standard deviations is maximized; 4) 5 point Slope Intercept method wherein a rolling 5 point slope and intercept (of fluorescence vs cycle) is used to predict the fluorescence for the next cycle and Cp cycle identified as the one where the % difference between the predicted fluorescence and the observed fluorescence Is maximized; 5) The maxRatio method where the PCR amplification signal is used to calculate a ratio at each cycle transforming the roughly sigmoidal shaped amplification curve to a ratio curve with a well-defined peak (Cp); and 6) Cy0 method where the Cy0 value is the intersection point between the abscissa axis and tangent of the inflection point of the Richards curve obtained by the non-linear regression of raw data.

The use of the term “Ct value” in this application is intended to encompass these alternatives.

Using the described approach, Ct values of various labeled amplification products from the target nucleic acid can be compared and used to distinguish wild-type amplification products from variant-containing amplification products. The method can be utilized to detect any variant or set of two adjacent or closely spaced variants, for example in distinguishing variant nucleotides in genes encoding antibiotic resistance mutations or for identifying disease-causing SNPs.

### I. EXEMPLARY DETECTION METHODS

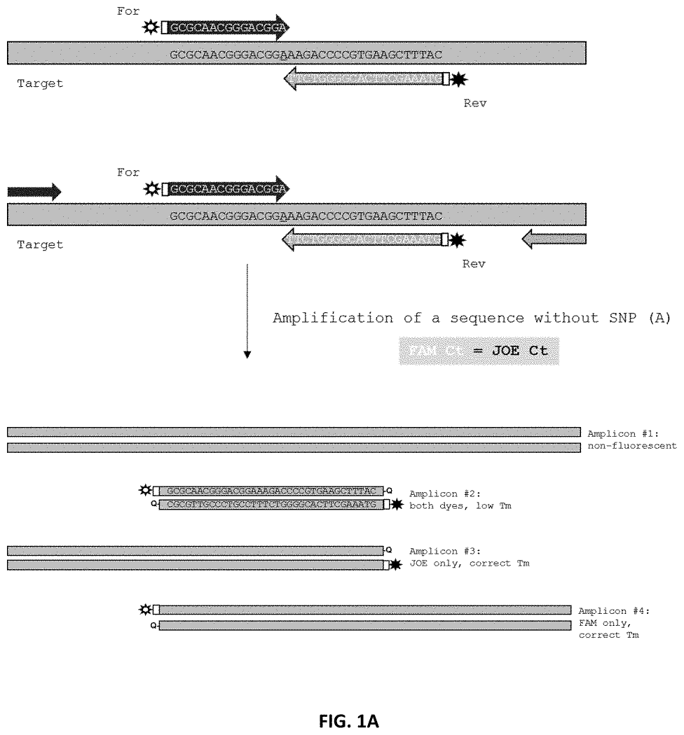

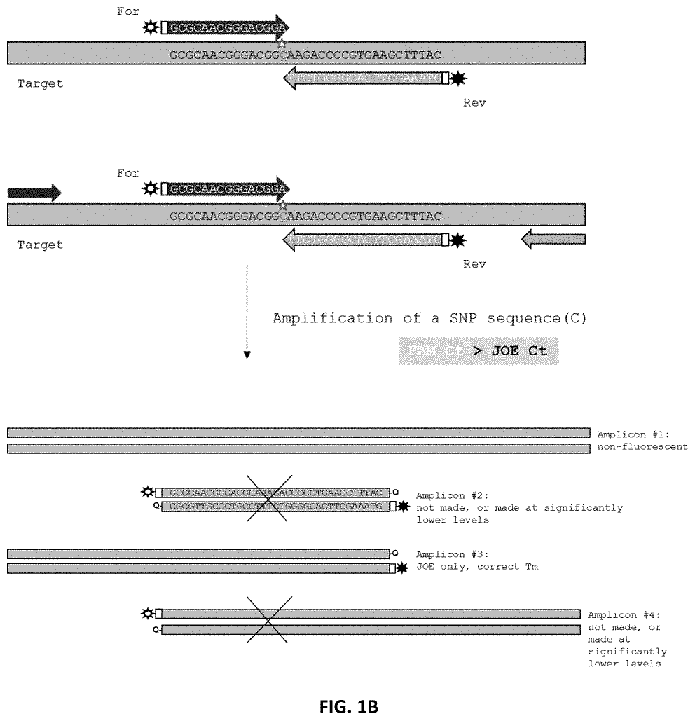

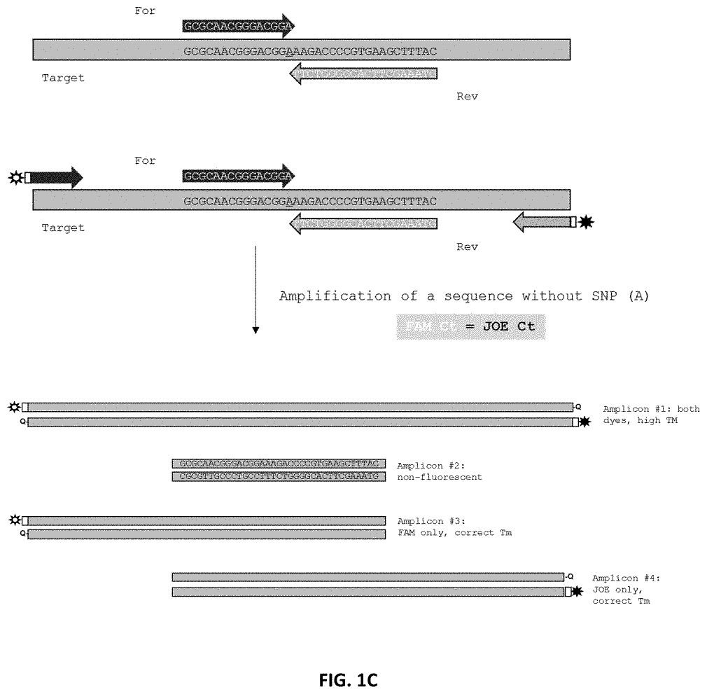

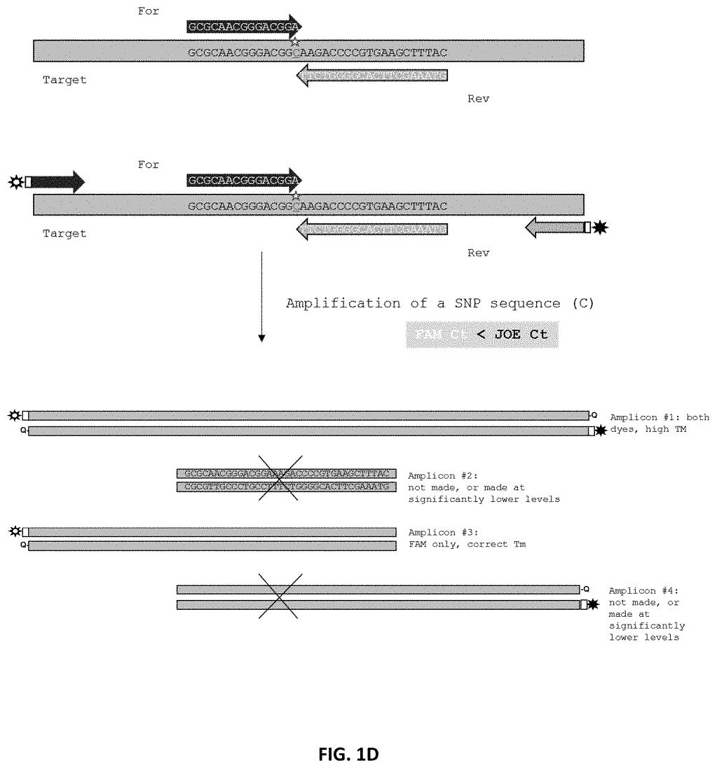

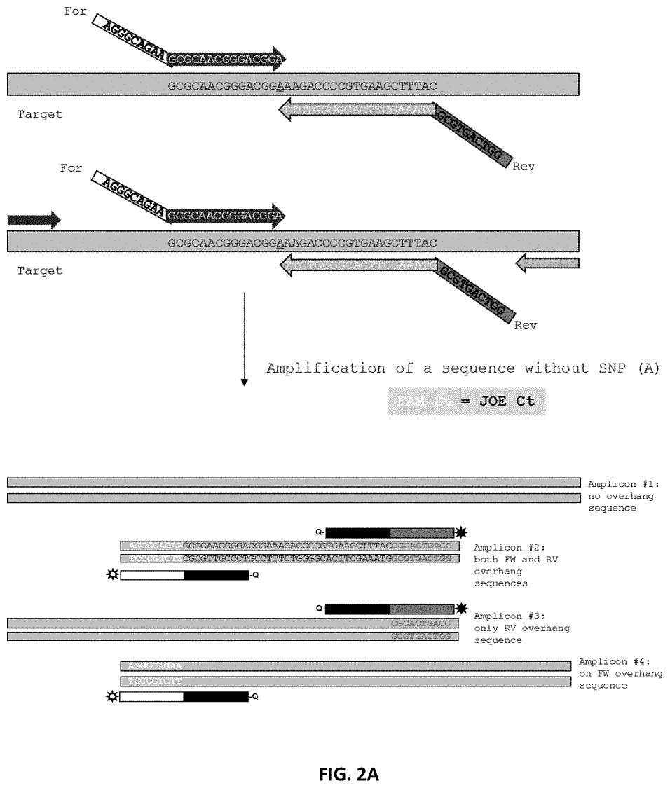

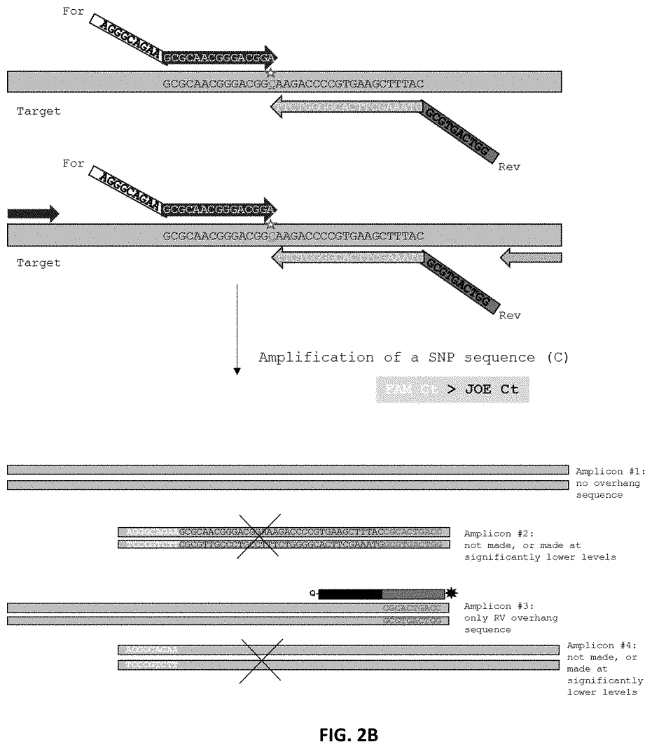

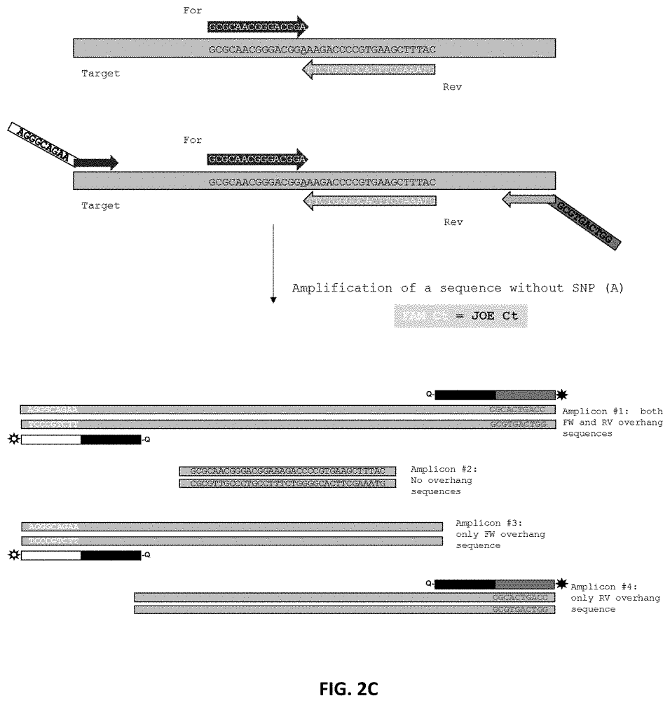

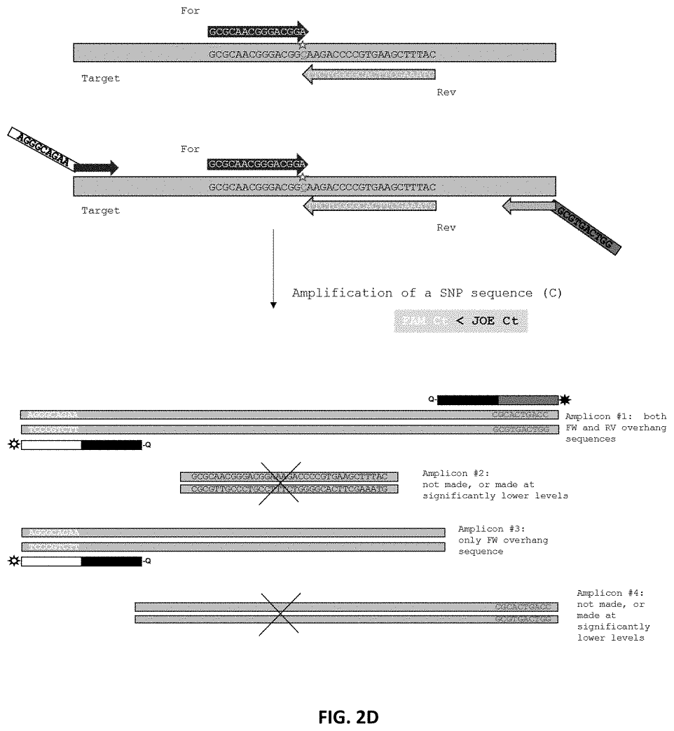

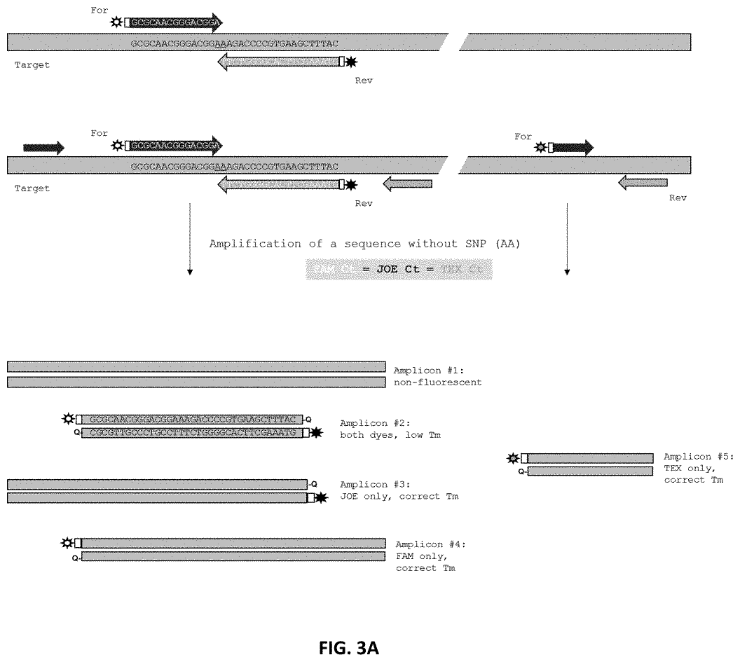

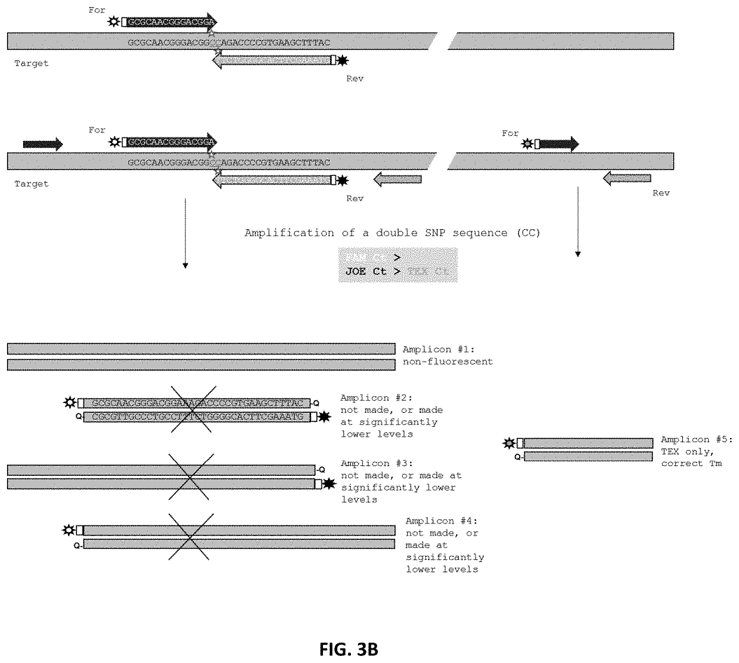

Various methods are described for performing the method, as shown in FIG. 1A-1D, FIGS. 2A-2D and 3A-3B. In an embodiment, the target nucleic acid has a first region and a second region, with the position of interest occurring in the first region. Two sets of primers are used to amplify the first and second regions of the target nucleic acid sequence. One primer of the first set of primers has a 3′ terminal nucleotide that is complementary to the wild-type nucleotide at the position of interest. One of the primers of the first primer set is labeled with a first signal-generating label so that amplification of the first region of the target nucleic acid by the first set of primers produces a labeled amplicon. The second set of primers is designed to amplify the second region of the target nucleic acid with the same, or similar efficiency as the first set of primers for amplifying the first region. One of the primers of the second set of primers is labeled with a second signal-generating label that is distinguishable from the first signal-generating label.

The first and second regions of the target nucleic acid can be within 500 nucleotides of each other, or within 300 nucleotides of each other. Preferably, the first region and the second region of the target sequence are within 200 nucleotides of each other so that primers from each of the first and second set can also form amplification products in co-operation with each other. In some embodiments, the first and second regions are within 150 nucleotides of each other, of within 100 nucleotides of each other, or within 80 nucleotides of each other, or within 60 nucleotides of each other.

The first and second regions of the target nucleic acid may partially overlap with each other (FIGS. 1A and 1B) or may be non-overlapping. It is however preferable that the primer binding sites are non-overlapping.

A reaction mixture comprising the first and second primer sets and the target nucleic acid is subjected to amplification conditions. Amplification may be monitored in real time as amplification proceeds and a Cycle threshold (Ct) value can be calculated for the various amplification products arising from the 4 primers in the reaction. As can be seen from FIG. 1A, 4 possible products may be produced from amplification of a target containing the wild-type nucleotide at the position of interest using two primer sets whose inner primers are designed to amplify overlapping segments of the target nucleic acid. In this scheme, only 3 of the products produce a detectable product as the longest amplicon is unlabeled. When the first and second region of the target nucleic acid are non-overlapping, only 3 amplification products would be produced as no amplification product is produced from the two inner primers. The embodiment shown in FIG. 1A uses MultiCode® RTx chemistry, in which each of the labels is coupled to a non-standard base at the 5′ end of the labeled primer and amplification is performed in the presence of complementary non-standard bases that hybridize only to their cognate non-standard bases. Typically, non-standard bases are isobases such as isoC and isoG. During polymerization, the complementary non-standard base will be incorporated into the amplicon only opposite the labeled non-standard base. If the labeled base is labeled with a flour, incorporation of a complementary non-standard base coupled to a quencher results in quenching of the fluorescent signal. In this embodiment, the fluorescent signal associated with amplification decreases as amplification proceeds, permitting detection of the change in signal and calculation of a Ct value associated with each label. Of the 3 detectable amplification products in FIG. 1A, one is labeled with both fluors (FAM and JOE), one is labeled with the first fluor (FAM) and one is labeled with the second fluor (JOE). Assuming equivalent amplification efficiency for all 3 products, one would expect Ct values for all 3 products to be approximately equal in targets having a wild-type nucleotide at the position of interest. While it is preferable that amplification efficiency is equivalent for all amplicons, it is not essential as a maximum threshold difference between fluor-specific Ct values can be determined for wild-type target nucleic acid sequences. Under the conditions tested, the inventors found that Ct values for amplification products produced using the method in wild-type target nucleic acids differ by no more than 3 Cts. However, under different assay conditions the maximum threshold for Ct differences between amplification products resulting from amplification of wild type targets may be less than three or more than three, and should be established for each set of conditions.

In the presence of a variant nucleotide at the position of interest however, amplification from the wild-type allele-specific primer will be significantly diminished, resulting in significantly greater or undetectable Ct values. Essentially, only 1 detectable product having a Ct value similar to that achieved using a wild type target is produced (FIG. 1B). Comparison of Ct values associated with the labeled amplification products in this scenario will yield a difference that is representative of the much less efficient and possibly non-detectable amplification from the wild type allele-specific primer. The inventors have found that under the conditions tested, targets having variant nucleotides at the position of interest showed a difference of 8 Cts or greater than Ct values obtained for targets having wild-type nucleotides at the position of interest.

By comparing the difference in Ct values obtained for amplification products from wild-type sequences with those from an unknown target nucleic acid, it is possible to determine whether a variant or wild-type sequence has been amplified from the unknown target nucleic acid. Since the allele-specific primer is specific to the wild-type nucleotide at the position of interest, the method will detect any variant nucleotide at this position, regardless of the actual nucleotide at the position of interest.

FIGS. 1C and 1D illustrate an alternate labeling scheme that may be used in the method of the invention. In this scenario, the first and second regions may also be partially overlapping and the outermost primers of the two sets of primers are labeled, such that the longest amplification product arising from extension of the two outermost primers is labeled and the shortest amplification product arising from extension of the two innermost primers is unlabeled. As for the labeling scheme shown in FIGS. 1A and 1B, wild-type target nucleic acid will yield roughly equivalent Ct values for all 3 detectable amplicons (FIG. 1C), while variant-containing target nucleic acids will yield significantly increased or undetectable Ct values for 1 of 3 detectable amplicons (FIG. 1D). By comparing the difference in Ct values determined for differentially labeled products, it is possible to distinguish between wild-type and variant nucleotides at the position of interest. Those skilled in the art will recognize that other primer labeling schemes suitable for use in real time detection may also be used with the method of the invention. For example, Lux™ primers, Scorpion® primers and Ampliflour® primers would be suitable for use in the method.

In the embodiments shown in FIG. 1A-2B, the wild-type allele-specific primer is positioned so that the 3′ terminal nucleotide corresponds to the position of the nucleotide of interest. A person skilled in the art will recognize that allele-specific amplification also occurs under conditions in which the allele-specific primer binds at a position that results in a mismatch at the nucleotide immediately upstream of the terminal nucleotide (i.e. at the n-1 position), or two (n-2) or three (n-3) nucleotides upstream of the terminal nucleotide. In some instances, both the terminal (n) nucleotide and the immediate upstream (n-1) nucleotides show mismatched binding to variant target nucleic acids. Generally, the wild type allele-specific primer should be designed to bind to a target nucleic acid having a wild type nucleotide at the position of interest with a Tm that is at least 3° C. higher than the Tm when bound to a target nucleic acid having a variant nucleotide at the position of interest.

FIG. 2A illustrates an alternative labeling scheme for detection of amplification products that uses tagged primers and labeled probes to detect amplification products. Each set of primers includes one primer that has a unique 5′ tag sequence that is not complementary to the target nucleic acid and preferably not complementary to any nucleic acid in the reaction mixture, and a 3′ portion that is complementary to the target nucleic acid. One set of primers includes an allele-specific primer having a 3′ terminal nucleotide that is complementary to the wild-type nucleotide at the position of interest. The unique 5′ tag sequence gets incorporated into the extended strand that serves as a template for second strand synthesis during PCR amplification. Amplification is performed in the presence of two distinguishably labeled probes for real time detection of amplification. Each different probe is designed to hybridize to extension products containing a complement of a unique 5′ tag sequence and different probes are labeled with distinguishable fluorophores, for example, FAM and JOE. Thus, in FIG. 2A, the FAM-labeled probe has a sequence that is sufficiently complementary to hybridize to the complement of the forward tagged inner primer and the JOE-labeled probe has a sequence that is sufficiently complementary to hybridize to the complement of the tagged reverse inner primer. The probes may be designed to hybridize to a region encompassing the entire complement of the tagged primer sequence, or only a portion of the tagged primer sequence, provided the probe binds specifically to the complement of the tagged primer sequence. Each probe is thus capable of binding to amplicons containing a sequence complementary to a respective inner primer. In general, the probes are designed to specifically hybridize to the complement of a sequence that includes both a tag sequence and a portion of the target sequence. Probes that exhibit a change in signaling properties depending on whether they are bound to target nucleic acids or not are suitable for use in the method. Suitable probes may be those described in Lukhtanov et al., 2007, NAR 35(5): e30, having a fluor at one end and a quencher attached at the other end. In the absence of target, such probes form a random coil, bringing the fluor and quencher into close proximity resulting in quenching of fluorescent signal. In the presence of target the probes hybridize to a complementary sequence, resulting in separation of the fluor and quencher and resultant fluorescent signal. Those skilled in the art will recognize that other probes useful for real time PCR detection, for example hydrolysis probes such as Taqman® probes, Scorpion® Probes, Molecular Beacons® and probes such as those described in US20160040219 are also suitable for use in the method of the invention.

Probe sequences are preferably the same as the tagged primer sequences to avoid hybridization to primers and permit specific hybridization extended amplicons arising from extended tagged primer sequences. It is not essential however that probe sequences are identical to tagged primer sequences, but they should be sufficiently complementary to a complement of the tagged primer sequence to bind thereto under stringent hybridization conditions. Probe sequences may be designed to be only partially complementary to tagged primer complements, either due to being identical to the 5′ tag sequence and only a portion of the 3′ target-specific region of the tagged primer, or due to having some non-identical nucleotides.

In the embodiment shown in FIG. 2A, amplification of a template containing a wild-type nucleotide produces 3 detectable amplicons resulting from different combinations of the 4 primers in the reaction. Amplification from the two outermost primers will yield the longest amplicon, however this amplicon will not be detected by the labeled probe since it does not include sequences complementary to the unique 5′ tag sequences of the primers. Two amplicons each include a region that is complementary to the full length of one of the two different probes, since each of these amplicons results from amplification using one or the other inner primer having a unique 5′ tag sequence and a respective outer primer. Assuming equivalent amplification efficiency for these two amplicons, each amplicon will be detected at approximately equivalent Ct values by the corresponding probe. The shortest amplicon is the result of amplification using the innermost primers and thus contains both unique 5′ tag primer sequences, each detectable by one of the distinguishably labeled probes. This amplicon will thus display equivalent Ct values in each detection channel for a target having a wild type nucleotide at the position of interest. As noted for the previous embodiment using labeled primers, even if Ct values calculated for the various amplicons are not equivalent as a result on non-equivalent amplification efficiency, a maximum threshold difference between Ct values for each probe can be established for wild-type sequences for comparison with unknown samples.

In contrast, amplification of a target sequence containing a variant nucleotide at the position of interest results in altered ratios of the 4 possible amplicons (FIG. 2B). Amplicons resulting from amplification using a tagged inner primer having a mismatched 3′ terminal nucleotide due to the presence of a variant nucleotide at the position of interest will be produced in significantly reduced or undetectable amounts. Therefore, Ct values arising from probes designed to detect wild type amplicons will be significantly increased or not detected. Accordingly, Ct values obtained for the two different fluors will not be equivalent. Thus, a comparison of Ct values for each of the two distinguishably labeled probes allows determination of whether the target sequence has a wild-type or variant nucleotide at the position of interest based on whether the difference in Ct values is above or below the threshold established for wild-type sequences.

FIGS. 2C and 2D illustrate an alternate arrangement in which the 5′ tags are attached to the outermost primers rather than the innermost primers. In this embodiment, probes are designed to detect amplicons arising from extension of the outer primers, thus probes include at least a 5′ portion that will hybridize to tag complementary sequences. Probes may also include 3′ target-specific sequences of the tagged primers for specific hybridization to extension products. As in the previous embodiment, amplification of target sequences having a wild-type nucleotide at the position of interest produces 3 detectable amplicons with approximately equivalent Ct values detected for each of the two distinguishably labeled probes. In contrast, amplification of target sequences having a variant nucleotide at the position of interest produces only 2 of the possible 3 detectable amplicons in equivalent amounts. Amplicons resulting from inner primers having a mismatch at the 3′ terminal nucleotide are produced at significantly reduced or undetectable levels. Variant nucleotides at the position of interest thus result in significantly increased/undetectable Ct values for these amplicons. As described for other embodiments, a comparison of the difference in Ct values calculated for the different reporters and determination of whether the difference is above or below a threshold established for wild-type targets enables the determination of whether a wild type or variant nucleotide is present at the position of interest.

The method of the invention may also be used in the detection of variant nucleotides at adjacent, or closely-spaced (within 15-20 nucleotides of each other) first and second positions of interest (FIGS. 3A&B). Because the method requires primers for detecting only wild-type nucleotides, it avoids the challenges of designing multiple primers that hybridize to overlapping target sequences for closely spaced nucleotides of interest. In this embodiment, wild-type allele-specific primers are designed for each of the first and second positions of interest and are designed to hybridize to different strands of the target nucleic acid to avoid overlapping primer hybridizing sites. Each wild-type allele-specific primer is labeled with a distinguishable label characteristic of the position of interest. Since neither wild-type allele-specific primer will generate an amplicon when paired with either the other wild-type inner allele-specific primer or a suitable outer primer in target nucleic acids having a variant nucleotide at both the first and second positions of interest, an additional primer set is included in the reaction mixture to serve as a control. One of the control primers is labeled with a label distinguishable from the other two labels in the reaction mixture and the primer set is designed to amplify a control target sequence that is upstream or downstream of the regions amplified by the first and second primer sets. Ideally the control primer set is designed to amplify the control target sequences with equivalent efficiency as the first and second primer sets, and targets a region far enough away from the target nucleic acid having the first and second positions of interest to ensure the control primer set does not amplify a region that overlaps with the regions amplified by the first and second primer sets.

The Ct value determined for the control amplicon serves as a comparator for Ct values determined for the amplicons containing the regions of interest. On a wild-type target nucleic acid, a comparison of Ct values associated with each of the three labels should show them to be approximately equivalent, or at least show a difference below a threshold value. This is because a wild-type template should result in amplification of the same 4 amplicons as previously described in FIG. 1A, with an additional control amplicon (FIG. 3A). However, when the target nucleic acid contains 2 adjacent or closely located variants at first and second positions of interest, only 2 amplicons will be produced—amplicon #1 which is unlabeled if the outer primer set is unlabeled, and amplicon #5 which is a positive control (FIG. 3B). In contrast to the scenario using wild-type template which shows approximately equivalent Ct values for all channels, target nucleic acids having variants at both nucleotides of interest will show greatly increased Ct values in both test channels compared to the control. Thus a comparison of the Ct value determined for the control amplicon with those containing variants at the positions of interest will show significantly higher or undetectable Cts for targets containing the variants.

### II. NUCLEIC ACIDS

As used herein “nucleic acid” means either DNA or RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, methylations, and unusual base-pairing combinations, such as the isobases. Accordingly, the nucleic acids described herein include not only the standard bases adenine (A), cytosine (C), guanine (G), thymine (T), and uracil (U) but also non-standard or non-natural nucleotides. Non-standard or non-natural nucleotides, which form hydrogen-bonding base pairs, are described, for example, in U.S. Pat. Nos. 5,432,272, 5,965,364, 6,001,983, 6,037,120, and 6,140,496, all of which are incorporated herein by reference. By “non-standard nucleotide” or “non-natural nucleotide” it is meant a base other than A, G, C, T, or U that is susceptible to incorporation into an oligonucleotide and that is capable of base-pairing by hydrogen bonding, or by hydrophobic, entropic, or van der Waals interactions, with a complementary non-standard or non-natural nucleotide to form a base pair. Some examples include the base pair combinations of iso-C/iso-G, K/X, K/P, H/J, and M/N, as illustrated in U.S. Pat. No. 6,037,120, incorporated herein by reference.

The hydrogen bonding of these non-standard or non-natural nucleotide pairs is similar to those of the natural bases where two or three hydrogen bonds are formed between hydrogen bond acceptors and hydrogen bond donors of the pairing non-standard or non-natural nucleotides. One of the differences between the natural bases and these non-standard or non-natural nucleotides is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-natural nucleotides for use in oligonucleotides include, for example, naphthalene, phenanthrene, and pyrene derivatives as discussed, for example, in Ren, et al., J. Am. Chem. Soc. 1996, 118:1671 and McMinn et al., J. Am. Chem. Soc. 1999, 121:11585, both of which are incorporated herein by reference. These bases do not utilize hydrogen bonding for stabilization, but instead rely on hydrophobic or van der Waals interactions to form base pairs.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, made up of “dNTPs,” which do not have a hydroxyl group at the 2′ position, and oligoribonucleotides, made up of “NTPs,” which have a hydroxyl group in the 2′ position. Oligonucleotides also may include derivatives, in which the hydrogen of the hydroxyl group is replaced with an organic group, e.g., an allyl group.

An oligonucleotide is a nucleic acid that includes at least two nucleotides. An oligonucleotide may be designed to function as a “primer.” A “primer” is a short nucleic acid, usually a ssDNA oligonucleotide, which may be annealed to a target polynucleotide by complementary base-pairing. The primer may then be extended along the target DNA or RNA strand by a polymerase enzyme, such as a DNA polymerase enzyme. Primer pairs can be used for amplification (and identification) of a nucleic acid sequence (e.g., by the polymerase chain reaction (PCR)). An oligonucleotide may be designed to function as a “probe.” A “probe” refers to an oligonucleotide, its complements, or fragments thereof, which are used to detect identical, allelic, or related nucleic acid sequences. Probes may include oligonucleotides that have been attached to a detectable label or reporter molecule. Typical labels include fluorescent dyes, quenchers, radioactive isotopes, ligands, scintillation agents, chemiluminescent agents, and enzymes.

An oligonucleotide may be designed to be specific for a target nucleic acid sequence in a sample. For example, an oligonucleotide may be designed to include “antisense” nucleic acid sequence of the target nucleic acid. As used herein, the term “antisense” refers to any composition capable of base-pairing with the “sense” (coding) strand of a specific target nucleic acid sequence. An antisense nucleic acid sequence may be “complementary” to a target nucleic acid sequence. As used herein, “complementarity” describes the relationship between two single-stranded nucleic acid sequences that anneal by base-pairing. For example, 5′-AGT-3′ pairs with its complement, 3′-TCA-5′. In some embodiments, primers or probes may be designed to include mismatches at various positions. As used herein, a “mismatch” means a nucleotide pair that does not include the standard Watson-Crick base pairs, or nucleotide pairs that do not preferentially form hydrogen bonds. The mismatch may include a natural nucleotide or a non-natural or non-standard nucleotide substituted across from a particular base or bases in a target. For example, the probe or primer sequence 5′-AGT-3′ has a single mismatch with the target sequence 3′-ACA-5′. The 5′ “A” of the probe or primer is mismatched with the 3′ “A” of the target. Similarly, the target sequence 5′-AGA-3′ has a single mismatch with the probe or primer sequence 3′-(iC)CT-5′. Here an iso-C is substituted in place of the natural “T.” However, the sequence 3′-(iC)CT-5′ is not mismatched with the sequence 5′-(iG)GA-3′.

Oligonucleotides may also be designed as degenerate oligonucleotides. As used herein, “degenerate oligonucleotide” is meant to include a population, pool, or plurality of oligonucleotides comprising a mixture of different sequences where the sequence differences occur at a specified position in each oligonucleotide of the population. Various substitutions may include any natural or non-natural nucleotide, and may include any number of different possible nucleotides at any given position. For example, the above degenerate oligonucleotide may instead include R=iC or iG, or R=A or G or T or C or iC or iG.

Oligonucleotides, as described herein, typically are capable of forming hydrogen bonds with oligonucleotides having a complementary base sequence. These bases may include the natural bases, such as A, G, C, T, and U, as well as artificial, non-standard or non-natural nucleotides such as iso-cytosine and iso-guanine. As described herein, a first sequence of an oligonucleotide is described as being 100% complementary with a second sequence of an oligonucleotide when the consecutive bases of the first sequence (read 5′-to-3′) follow the Watson-Crick rule of base pairing as compared to the consecutive bases of the second sequence (read 3′-to-5′). An oligonucleotide may include nucleotide substitutions. For example, an artificial base may be used in place of a natural base such that the artificial base exhibits a specific interaction that is similar to the natural base.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm, for example, nearest-neighbor parameters, and conditions for nucleic acid hybridization are known in the art.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, nucleoside triphosphates, and optionally, at least one labeled probe and/or optionally, at least one agent for determining the melting temperature of an amplified target nucleic acid (e.g., a fluorescent intercalating agent that exhibits a change in fluorescence in the presence of double-stranded nucleic acid).

The amplification methods described herein may include “real-time monitoring” or “continuous monitoring.” These terms refer to monitoring multiple times during a cycle of PCR, preferably during temperature transitions, and more preferably obtaining at least one data point in each temperature transition. The term “homogeneous detection assay” is used to describe an assay that includes coupled amplification and detection, which may include “real-time monitoring” or “continuous monitoring.”

Amplification mixtures may include natural nucleotides (including A, C, G, T, and U) and non-natural or non-standard nucleotides (e.g., including iC and iG). DNA and RNA oligonucleotides include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytosine (C), and uridine (U). These five bases are “natural bases.” According to the rules of base pairing elaborated by Watson and Crick, the natural bases hybridize to form purine-pyrimidine base pairs, where G pairs with C and A pairs with T or U. These pairing rules facilitate specific hybridization of an oligonucleotide with a complementary oligonucleotide.

The oligonucleotides and nucleotides of the disclosed methods may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the trade name “BHQ” (e.g., BHQ-0, BHQ-1, BHQ-2, and BHQ-3, Biosearch Technologies, Novato, Calif.). Dark quenchers also may include quenchers sold under the trade name “QXL™” (Anaspec, San Jose, Calif.). Dark quenchers also may include DNP-type non-fluorophores that include a 2-4-dinitrophenyl group.

### III. EXAMPLES

### 1) Wild-Type v. Variant Detection using Labeled Primers

Primers were designed to distinguish wild-type from each of two different variant nucleotides in a target. In this experiment, outer primers were labeled using distinguishable fluorescent dyes, similar to what is shown in FIGS. 1C and 1D. An allele-specific inner primer for each of the two different variants was designed to have its 3′ terminal nucleotide be complementary to the wild-type nucleotide at the position of interest. In this configuration, wild-type amplicons produced from the two outer primers, or one inner and one outer primer, are detectable since the outer primers are labeled. In contrast, amplicons produced from the two inner primers, are not detectable as these primers are not labeled.

A 25 μPCR reaction consisted of 10× ISOlution (Luminex Corp, Austin Tex.), 10 mM Tris, 2.5 mM Magnesium Chloride, 50 mM Potassium Chloride, 200 nM primer pairs (FW inside: GCGCAACGGGACGGA (SEQ ID No. 1), RV inside: GTAAAGCTTCACGGGGTCTT (SEQ ID No. 2), FW outside: /56-FAM//iMe-isodC/GACTCGGTGAAATCCAGGTA (SEQ ID No. 3), RV outside: /56-JOEN//iMe-isodC/ATGGTGGTGTTTTGATCAATATTA (SEQ ID No. 4)) and 1× glycerol-free Titanium Taq (Takara Bio U.S.A. Inc., Mountain View Calif.). Wild-type and variant sequences were obtained as gblocks (IDT), quantified, and diluted in 1M MOPS buffer (pH 7.5) with 0.5M EDTA (pH 8.0) to the appropriate concentrations. gBlock LH767, representing wild type, has an A at both positions of interest. gBlocks LH768, LH769 and LH770 have a G, C, and T at the first position respectively. gBlocks LH771, LH772, and LH773 have a G, C, and T at the second position respectively. 5 μL target nucleic acid was added to the PCR reaction to the appropriate number of copies/reaction as outlined below. Amplification was performed on the 7500 Real-Time PCR system (ThermoFisher) with the following cycling parameters: 50° C. for 5 minutes, 95° C. for 2 minutes and 20 seconds, followed by 45 cycles of denaturation at 95° C. for 10 seconds, annealing at 58° C. for 16 seconds. Melt analysis (95° C. to 60° C. to 95° C. at 0.5° C./sec) was performed to identify target amplicons. Data analysis was performed using the MultiCode-RTx software from Luminex Corporation.

From Table 1 below, it can be seen that an amplification reaction mixture comprising the 4 primers described above for amplifying a dilution series of wild-type target analyte (LH767), results in similar Ct values for FAM-labeled amplicons and JOE-labeled amplicons. The maximum difference in Ct for FAM-and JOE-labeled amplicons from wild-type targets was observed to be less than 2. In contrast, a dilution series of 2 different target analytes, each containing a variant nucleotide in one of two positions of interest targeted by two different inner primers, showed an increased Ct (8.5 Cts or more) for amplicons arising from the same primers when compared with wild-type target analytes. Specifically, amplicons generated from a first (sense) inner unlabeled primer in combination with a JOE-labeled (antisense) outer primer were detected at higher Ct values using targets LH768-770 than amplicons generated from a second (antisense) inner primer in combination with a FAM-labeled outer primer (Table 1). Targets LH768-770 are known to contain a variant nucleotide at the position of interest for the first inner primer. Similarly, amplicons generated from a second (antisense) inner primer in combination with a FAM-labeled outer primer were detected at higher Ct values using targets LH771-773 than amplicons generated from the first (sense) inner primer in combination with a JOE-labeled outer primer (Table 1). Targets LH771-773 are known to contain a variant nucleotide at the position of interest for the second inner primer.

TABLE 1
Ct values obtained for amplifying a dilution series of
wild-type target analyte sequence (LH767) and a dilution
series of variant target analyte sequence,
where the variant nucleotide is at a first position
of interest (LH768-770) or a second
position of interest (LH771-773). ND = Not detected.

| | FAM Ct | JOE Ct | Delta Ct |
|---|---|---|---|
| LH767 @ 3,000 copies/rxn | 28 | 30.3 | 2.3 |
| LH767 @ 300 copies/rxn | 31.9 | 33.8 | 1.9 |
| LH767 @ 30 copies/rxn | 36.9 | 39.9 | 3 |
| LH767 @ 3 copies/rxn | ND | ND | ND |
| LH768 @ 3,000 copies/rxn | 26.1 | 37.9 | 11.8 |
| LH768 @ 300 copies/rxn | 29.6 | ND | ND |
| LH768 @ 30 copies/rxn | 33 | ND | ND |
| LH768 @ 3 copies/rxn | 35.7 | ND | ND |
| LH769 @ 3,000 copies/rxn | 25.4 | 39.6 | 14.2 |
| LH769 @ 300 copies/rxn | 29.9 | ND | ND |
| LH769 @ 30 copies/rxn | 32.3 | ND | ND |
| LH769 @ 3 copies/rxn | 37.4 | 38.6 | ND |
| LH770 @ 3,000 copies/rxn | 26.1 | 41.6 | 15.5 |
| LH770 @ 300 copies/rxn | 30.2 | 43.3 | 13.1 |
| LH770 @ 30 copies/rxn | 33.4 | 41.9 | 8.5 |
| LH770 @ 3 copies/rxn | 37.2 | ND | ND |
| LH771 @ 3,000 copies/rxn | 35.1 | 25.3 | 9.8 |
| LH771 @ 300 copies/rxn | 39.1 | 28.7 | 10.4 |
| LH771 @ 30 copies/rxn | 41.5 | 32.3 | 9.2 |
| LH771 @ 3 copies/rxn | ND | 36.3 | ND |
| LH772 @ 3,000 copies/rxn | 36.4 | 24.8 | 11.6 |
| LH772 @ 300 copies/rxn | 40.2 | 29.3 | 10.9 |
| LH772 @ 30 copies/rxn | 40.3 | 32.3 | 8 |
| LH772 @ 3 copies/rxn | ND | 34.5 | ND |
| LH773 @ 3,000 copies/rxn | 36.3 | 25.2 | 11.1 |
| LH773 @ 300 copies/rxn | 40.3 | 28.5 | 11.8 |
| LH773 @ 30 copies/rxn | 41.2 | 32.7 | 8.5 |
| LH773 @ 3 copies/rxn | ND | 37 | ND |

### 2. Wild-Type v. Variant Detection using Labeled Probes

Primers were designed to distinguish wild-type from variant nucleotides at each of two nucleotide positions in a target nucleic acid sequence. In this experiment, inner primers (sense and antisense) were designed to be allele-specific, each having a 3′ terminal nucleotide complementary to the wild-type nucleotide at one of the positions of interest. Outer primers (sense and antisense) included a 5′ tag region that was not complementary to any other nucleic acids in the reaction. Each tag region was designed to generate an amplicon that could be specifically recognized by either a FAM-or JOE-labeled probe. In this configuration, wild-type amplicons produced from the two outer primers, or one outer and one inner primer, are detectable since the amplicons include anti-tag sequences that are recognized by one (or both) of the probes. In contrast, amplicons produced from the two inner primers, are not detectable because they lack anti-tag sequences.

The amplification reaction mixture included a first (sense) inner primer having a 3′ terminal nucleotide being complementary to a wild-type nucleotide at the first position of interest, a second (antisense) inner primer having a 3′ terminal nucleotide being complementary to a wild-type nucleotide at the second position of interest, a first (antisense) outer primer having a 5′ tag region specific for a JOE-labeled probe and a second (sense) outer primer having a 5′ tag region specific for a FAM-labeled probe.

A 25 82 l PCR reaction consisted of 1× standard PCR Buffer, 10 mM Tris, 2.5 mM Magnesium Chloride, 50 mM Potassium Chloride, 100/400 nM primer pairs (FW inside: GCGCAACGGGACGGA (SEQ ID No. 1), RV inside: GTAAAGCTTCACGGGGTCTT (SEQ ID No. 2), FW outside: GGCTGACTGCGGACTCGGTGAAATCCAGGTA (SEQ ID No. 5), RV outside: CTTCAGCAATCCTCTACATGGTGGTGTTTTG (SEQ ID No. 6), 100 nM each probe (AP525-MGB-cttcagca*a*tcctcta*ca (SEQ ID No. 7), and FAM-MGB-ggctga*ctgcggactcgg (SEQ ID No. 8)) (where * denotes superbases) and 1× glycerol-free Titanium Taq (Takara Bio U.S.A Inc., Mountain View Calif.). Target gBlocks were prepared and utilized as outlined above. Cycling parameters were as described for Example 1 were used except for the omission of the melt step.

Table 2 shows that Ct values obtained using FAM-and JOE-labeled probes containing distinct 5′ tag regions were approximately equivalent for a dilution series of target nucleic acid having wild-type nucleotides at both positions of interest (LH767). Furthermore, both probes were capable of detecting the amplicons at similar LoD of between 150-300 copies/rxn. In contrast, Ct values from a dilution series of 3 target analytes containing a variant at the first position of interest (LH768-770) were increased by at least 9.7 Cts using JOE-labeled probes relative to the FAM probe (Table 2). Similarly, Ct values from a dilution series of 3 target analytes containing a variant at the second position of interest (LH771-773) were increased by at least 8.3 Cts using the FAM probe relative to the JOE probe (Table 2).

From the results presented herein, the labeled primer method appears to be roughly 10-fold more sensitive than the probe method. However, utilizing a probe provides an added level of specificity, making melt analysis unnecessary.

TABLE 2
Ct values obtained for amplifying a dilution series of target
analytes containing no variants (LH767),
a dilution series of target analytes containing
a variant at the first position of interest
(LH768-770) and a dilution series of target analytes
containing a variant at the second position of
interest (LH771-773). ND = Not detected.

| | FAM Ct | JOE Ct | Delta Ct |
|---|---|---|---|
| LH767 @ 3,000 copies/rxn | 31.4 | 31 | 0.4 |
| LH767 @ 300 copies/rxn | 34.9 | 36 | 1.1 |
| LH767 @ 30 copies/rxn | ND | ND | ND |
| LH768 @ 3,000 copies/rxn | 28.9 | 38.6 | 9.7 |
| LH768 @ 300 copies/rxn | 33 | ND | ND |
| LH768 @ 30 copies/rxn | 38.1 | ND | ND |
| LH769 @ 3,000 copies/rxn | 28.2 | ND | ND |
| LH769 @ 300 copies/rxn | 33 | ND | ND |
| LH769 @ 30 copies/rxn | 39 | ND | ND |
| LH770 @ 3,000 copies/rxn | 28.4 | 44 | 15.6 |
| LH770 @ 300 copies/rxn | 33 | ND | ND |
| LH770 @ 30 copies/rxn | 37.9 | ND | ND |
| LH771 @ 3,000 copies/rxn | 34.9 | 26.6 | 8.3 |
| LH771 @ 300 copies/rxn | 40 | 30.6 | 9.4 |
| LH771 @ 30 copies/rxn | ND | 35.4 | ND |
| LH772 @ 3,000 copies/rxn | 36.9 | 26.5 | 10.4 |
| LH772 @ 300 copies/rxn | 41.2 | 31 | 10.2 |
| LH772 @ 30 copies/rxn | ND | 35 | ND |
| LH773 @ 3,000 copies/rxn | 35.8 | 26.4 | 9.4 |
| LH773 @ 300 copies/rxn | 39.2 | 30 | 9.2 |
| LH773 @ 30 copies/rxn | 43.9 | 35.4 | 8.5 |

1. A method of determining the presence of a wild type or variant nucleotide at a position of interest in a target nucleic acid sequence, the target nucleic acid sequence having first and second regions and the position of interest being located in the first region, the method comprising the steps of:
a) providing a first primer pair capable of specific amplification of the first region of the target nucleic acid sequence if present, to form a first amplicon, wherein one primer of the pair has a 3′ terminal nucleotide that is complementary to the wild-type nucleotide at the position of interest and wherein the first amplicon is labeled with a first signal-generating label coupled to one of the primers of the first pair of primers;
b) providing a second primer pair capable of specific amplification of the second region of the target nucleic acid sequence, if present, to form a second amplicon, wherein the second amplicon is labeled with a second signal-generating label coupled to one of the primers of the second pair of primers;
c) forming a reaction mixture comprising the first and second pairs of primers, and the target nucleic acid under conditions for nucleic acid amplification;
d) measuring first and second signals from each of the first and second labels as amplification proceeds and calculating a cycle threshold (Ct) value associated with each of the first and second signals;
e) comparing the Ct values associated with the first and second signals; and
f) determining the presence of a wild-type nucleotide at the position of interest if the difference between the Ct values associated with the first and second signals is less than or equal to a predetermined threshold or determining the presence of a variant nucleotide at the position of interest if the difference between the Ct values associated with the first and second signals is greater than the predetermined threshold.

2. The method of claim 1 wherein the first and second regions of the target nucleic acid partially overlap.

3. The method of claim 1 wherein the first and second regions of the target nucleic acid do not overlap.

4. The method of claim 1 wherein the first and second regions of the target nucleic acid are within 500 nucleotides of each other, or within 300 nucleotides of each other, or within 200 nucleotides of each other, or within 150 nucleotides of each other, or within 100 nucleotides of each other, or within 80 nucleotides of each other.

5. (canceled)

6. The method of claim 1 wherein the first and second signal-generating labels are coupled to a non-standard base at a 5′ end of each primer and wherein amplification results in the incorporation of a complementary non-standard base opposite the non-standard base of each primer.

7. The method of claim 6 wherein the non-standard base is one of isoC or isoG.

8. (canceled)

9. The method of claim 6 wherein the first and second signal-generating labels are distinguishable fluorophores, and the complementary non-standard base is coupled to a quencher.

10. The method of claim 1 wherein the predetermined threshold is the difference in Ct values associated with the first and second signals from a target nucleic acid having a wild type nucleotide at the position of interest.

11. A method of determining the presence of a wild-type or variant nucleotide at each of first and second positions of interest in a target nucleic acid, wherein the first and second positions of interest are within 15-20 nucleotides of each other, the method comprising:
a) providing a first primer pair capable of specific amplification of a first portion of the target nucleic acid to form a first amplicon, wherein one primer of the pair has a 3′ terminal nucleotide that is complementary to the wild-type nucleotide at the first position of interest and wherein the first amplicon is labeled with a first signal-generating label coupled to one of the primers of the first pair of primers;
b) providing a second primer pair capable of specific amplification of a second portion of the target nucleic acid to form a second amplicon, wherein one primer of the second primer pair has a 3′ terminal nucleotide that is complementary to the complement of the wild-type nucleotide at the second position of interest, and wherein the second amplicon is labeled with a second signal-generating label coupled to one of the primers of the second pair of primers;
c) providing a third primer pair capable of specific amplification of a third portion of the target nucleic acid, if present, to form a third amplicon, wherein the third amplicon does not overlap with the first or second amplicon, and wherein the third amplicon is labeled with a third signal-generating label coupled to one of the primers of the third pair of primers;
d) forming a reaction mixture comprising the first, second and third pairs of primers, and the target nucleic acid under conditions for nucleic acid amplification;
e) measuring signal from each of the first, second and third labels as amplification proceeds and calculating a Ct value associated with each of the first, second and third signals;
f) comparing the Ct values associated with the first and third signals and comparing the Ct values associated with the second and third signals; and
g) determining the presence of the wild-type nucleotide at the first position of interest if the difference between the Ct values associated with the first and third signals is less than or equal to a first predetermined threshold, determining the presence a variant nucleotide at the first position of interest if the difference between the Ct values associated with the first and third signals is greater than the first predetermined threshold, determining the presence of the wild-type nucleotide at the second position of interest if the difference between the Ct values associated with the second and third labels is less than or equal to a second predetermined threshold or determining the presence of a variant nucleotide at the second position of interest if the difference between the Ct values associated with the second and third labels is greater than the second predetermined threshold.

12. The method of claim 11 wherein the first and second portions of the target nucleic acid overlap.

13. (canceled)

14. The method of claim 11 wherein the first, second and third signal-generating labels are coupled to a non-standard base at a 5′ end of each primer and wherein amplification results in the incorporation of a complementary non-standard base opposite the non-standard base of each primer.

15. The method of claim 14 wherein the non-standard base is one of isoC or isoG.

16. (canceled)

17. The method of claim 16 wherein the first, second and third signal-generating labels are distinguishable fluorophores, and the complementary non-standard base is coupled to a quencher.

18. The method of claim 11 wherein the first predetermined threshold is the difference in Ct values associated with the first and third signals from a target nucleic acid having a wild type nucleotide at the first position of interest and the second predetermined threshold is the difference in Ct values associated with the second and third signals from a target nucleic acid having a wild type nucleotide at the second position of interest.

19.-36. (canceled)

37. A method of determining the presence of a wild-type or variant nucleotide at a position of interest, in a target nucleic acid, the target nucleic acid having first and second regions and the nucleotide of interest being located in the first region, the method comprising the steps of:
a) providing a first primer pair capable of specific amplification of the first region of the target nucleic acid to form a first amplicon, wherein one primer of the pair is an allele-specific primer and has a Tm that is at least 3° C. degrees higher when hybridized to a target nucleic acid having a wild-type nucleotide at the position of interest than when hybridized to a target nucleic acid having a variant nucleotide at the position of interest, and wherein the first amplicon is labeled with a first signal-generating label coupled to one of the primers of the first pair of primers;
b) providing a second primer pair capable of specific amplification of the second region of the target nucleic acid to form a second amplicon, wherein the second amplicon is labeled with a second signal-generating label coupled to one of the primers of the second pair of primers;
c) forming a reaction mixture comprising the first and second pairs of primers, and the target nucleic acid under conditions for nucleic acid amplification;
d) measuring first and second signals from each of the first and second signal-generating labels as amplification proceeds and calculating first and second Ct values associated with each of the first and second signals; and
e) comparing the first and second Ct values and determining the presence of a wild-type nucleotide at the position of interest if the difference between the first and second Ct values is less than or equal to a predetermined threshold or determining the presence of a variant nucleotide at the position of interest if the difference between the first and second Ct values is greater than the predetermined threshold.

38. The method of claim 37 wherein the allele-specific primer hybridizes to the target nucleic acid such that the position of interest corresponds to a 3′ terminal nucleotide of the primer, or the position of interest corresponds to the nucleotide immediately upstream of a 3′ terminal nucleotide of the primer or the position of interest corresponds to the nucleotide two positions upstream of a 3′ terminal nucleotide of the primer.

39.-44. (canceled)

45. The method of claim 37 wherein the first and second signal-generating labels are coupled to a non-standard base at a 5′ end of each primer and wherein amplification results in the incorporation of a complementary non-standard base opposite the non-standard base of each primer.

46. The method of claim 45 wherein the non-standard base is one of isoC or isoG.

47. (canceled)

48. The method of claim 45 wherein the first and second signal-generating labels are distinguishable fluorophores, and the complementary non-standard base is coupled to a quencher.

49. The method of claim 45 wherein the predetermined threshold is the difference in Ct values associated with the first and second signals from a target nucleic acid having a wild type nucleotide at the position of interest.

50. (canceled)
